(19) United States
(12) Patent Application Publication
(10) Pub. No.: US 2025/0268491 A1
(43) Pub. Date: Aug. 28, 2025

(54) TRANSDERMAL GLUCOSE MONITOR COVER

(71) Applicant: T D' Avignon, Missouri City, TX (US)

(72) Inventor: T D' Avignon, Missouri City, TX (US)

(21) Appl. No.: 18/588,287

(22) Filed: Feb. 27, 2024

(51) Int. Cl. A61B 5/145

(52) U.S. Cl. CPC A61B 5/14532; A61B 5/14503

(57) ABSTRACT

A cover apparatus for obstructing a transdermal glucose monitor from view includes a top wall shaped and a perimeter wall. The perimeter wall is coupled to and extends away from the top wall. The perimeter wall and the top wall define a cavity which is dimensioned and configured to receive the transdermal glucose monitor therein

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to cover and more particularly pertains to a new cover for obstructing a transdermal glucose monitor from view.

### (2) Description of Related Art Including Information Disclosed Under 37 CFR 1.97 and 1.98

The prior art describes devices for attaching a transdermal glucose monitor to a user's skin. However, the prior art fails to disclose a cover for obscuring the transdermal glucose monitor from view, and either blending visually with the user's skin or ornamenting it.

### BRIEF SUMMARY OF THE INVENTION

An embodiment of the disclosure meets the needs presented above by generally comprising a top wall shaped and configured to cover a top surface of a transdermal glucose monitor. A perimeter wall is coupled to and extends away from the top wall. The perimeter wall and the top wall define a cavity which is dimensioned and configured to receive the transdermal glucose monitor therein.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a bottom perspective view of a cover apparatus according to an embodiment of the disclosure.

FIG. 2 is a top perspective view of an embodiment of the disclosure.

FIG. 3 is an exploded view of an embodiment of the disclosure in-use with a transdermal glucose monitor.

FIG. 4 is an in-use view of an embodiment of the disclosure.

FIG. 5 is an in-use view of an embodiment of the disclosure.

FIG. 6 is a bottom perspective view of an embodiment of the disclosure.

FIG. 7 is an exploded side view of an embodiment of the disclosure in-use with a transdermal glucose monitor.

### DETAILED DESCRIPTION OF THE INVENTION

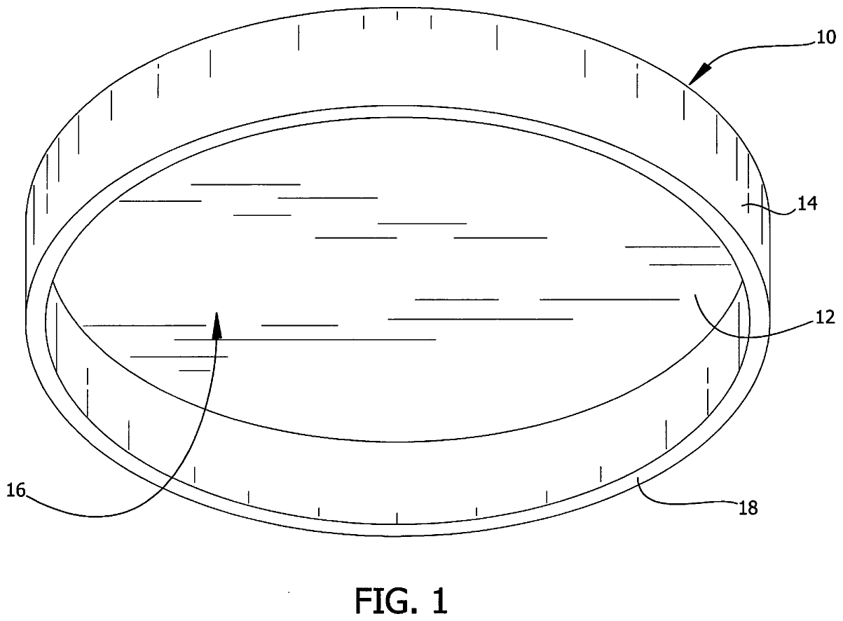

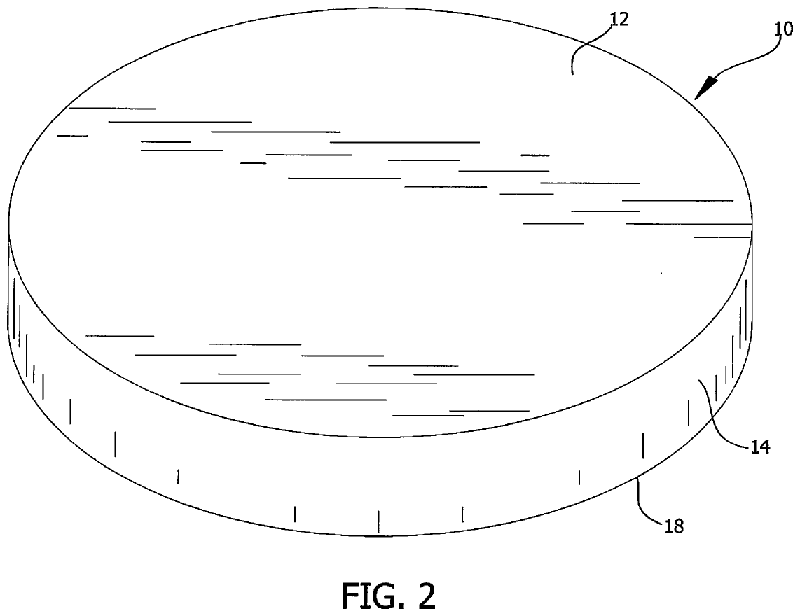

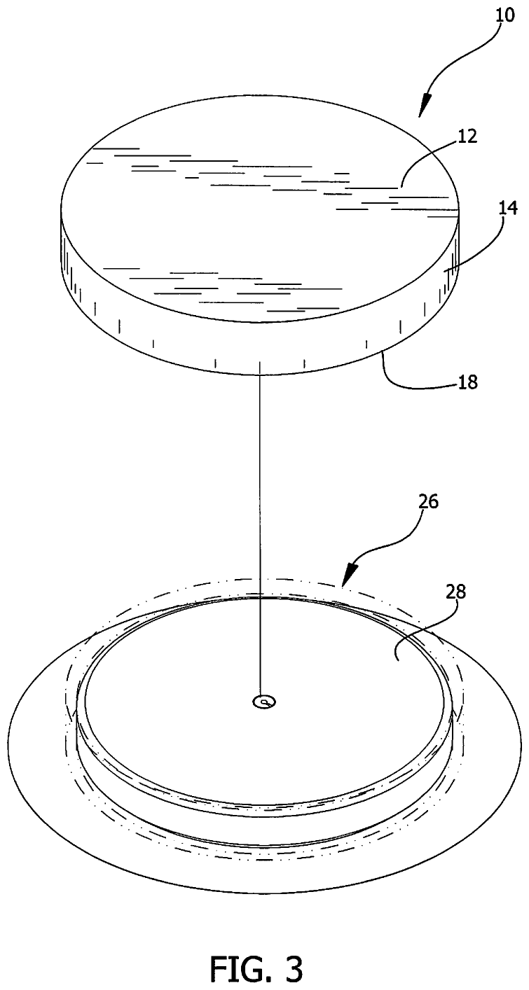

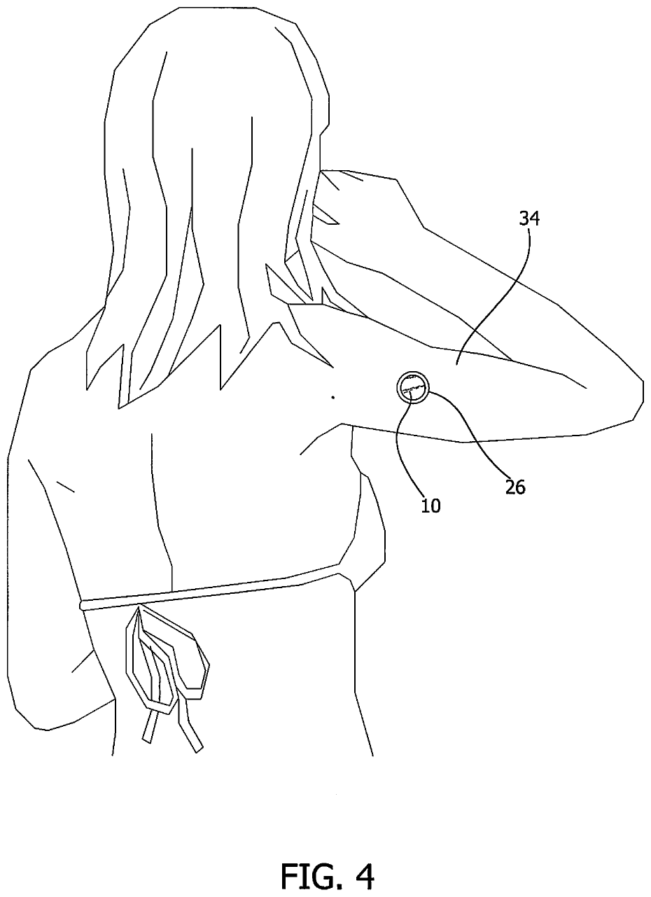

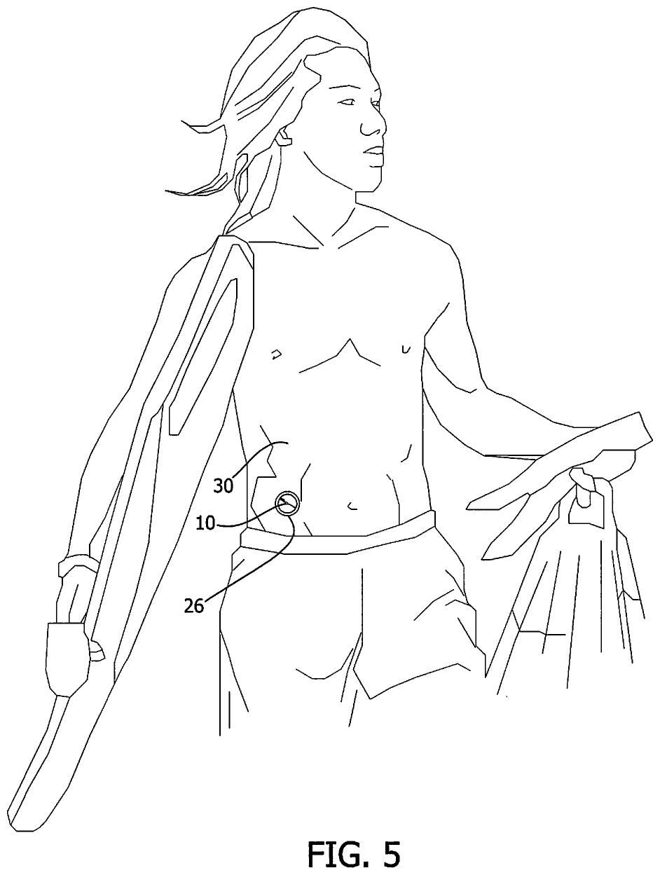

With reference now to the drawings, and in particular to FIGS. 1 through 7 thereof, a new cover embodying the principles and concepts of an embodiment of the disclosure and generally designated by the reference numeral 10 will be described.

As best illustrated in FIGS. 1 through 7, the cover apparatus 10 generally comprises a top wall 12 and a perimeter wall 14. The perimeter wall 14 is coupled to and extends perpendicularly away from the top wall 12. The top wall 12 and the perimeter wall 14 define a cavity 16 which is dimensioned and configured to receive a transdermal glucose monitor 26 which is attached to a user's skin 34. The transdermal glucose monitor 26 has a disc shape, and the top wall 12 has a circular shape such that the top wall 12 covers a top surface 28 of the transdermal glucose monitor 26. The top wall 12 and the perimeter wall 14 may have different shapes in other embodiments such that the cover apparatus 10 may surround transdermal glucose monitor 26s of other shapes. The top wall 12 and the perimeter wall 14 have uniform thicknesses and are integrally formed with each other.

In reference to FIGS. 1 through 3, the perimeter wall 14 of one embodiment comprises a resiliently stretchable material such that the perimeter wall 14 stretches to accommodate the transdermal glucose monitor 26. The perimeter wall 14 then grips the transdermal glucose monitor 26 to keep the cover apparatus 10 mounted to the transdermal glucose monitor 26.

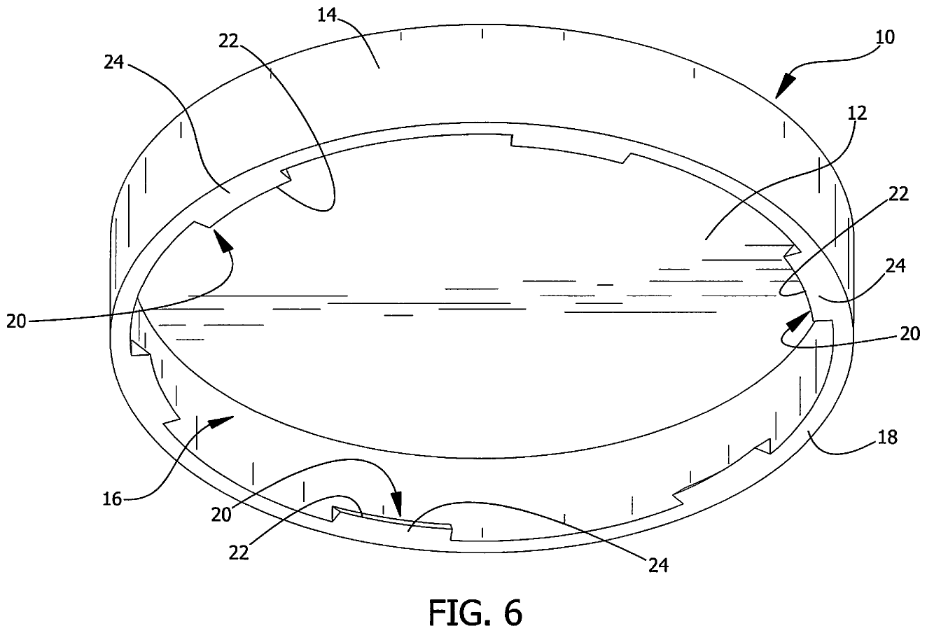

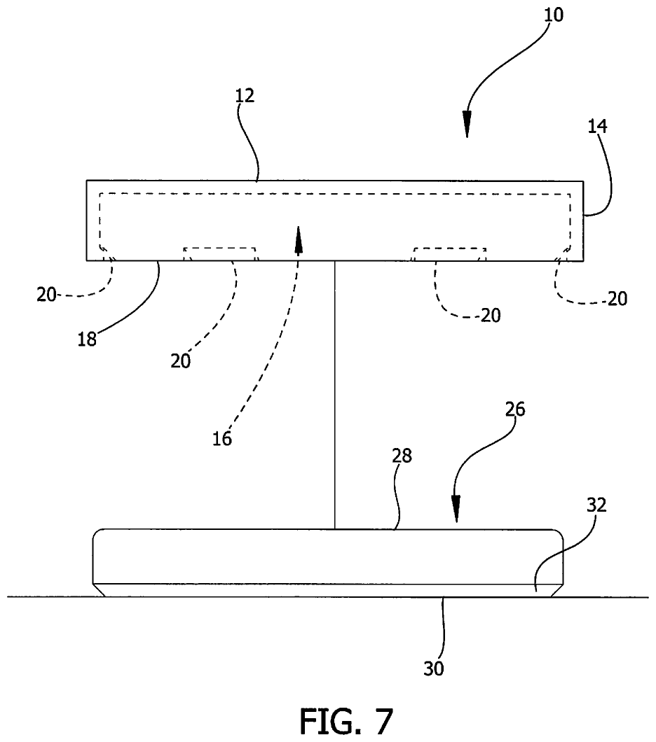

Another embodiment is depicted in FIGS. 6 and 7 which further comprises a plurality of protrusions 20 that are coupled to and extend inwardly from the perimeter wall 14. The protrusions 20 are positioned distally from the top wall 12 and are shaped to seat on a shoulder 32 of the transdermal glucose monitor 26 at a bottom end 30 of the transdermal glucose monitor 26. The protrusions 20 are radially spaced from each other along the perimeter wall 14. Each protrusion 20 tapers from the perimeter wall 14 to a distal edge 22 with respect to the perimeter wall 14. Each protrusion 20 also has a lower surface 24 which is coplanar with a lower end 18 of the perimeter wall 14. The protrusions 20 may be integrally formed with the perimeter wall 14. This embodiment is configured to have a snap connection with the transdermal glucose monitor 26.

Other embodiments of the present disclosure may have other connectors for attaching the cover apparatus 10 to the transdermal glucose monitor 26 such as latches, hook-and-loop fasteners, zippers, or the like. The cover apparatus 10 may be colored to blend in with the user's skin 34 or may be ornamented with a visual design, photograph, or the like. The cover apparatus 10 may be constructed of silicone, rubber, plastic, or any other suitable material.

In use, the cover apparatus 10 is attached to the transdermal glucose monitor 26 to obstruct the transdermal glucose monitor 26 from view. The cover apparatus 10 may be colored to camouflage the transdermal glucose monitor 26 on the user's skin 34 or may be designed to ornament the user. Ornamental designs may comprise geometric patterns, photographs, logos, symbols, or the like.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A cover apparatus for a transdermal glucose monitor, the apparatus comprising:
a top wall shaped and configured to cover a top surface of the transdermal glucose monitor; and
a perimeter wall coupled to and extending away from the top wall, the perimeter wall and the top wall defining a cavity dimensioned and configured to receive the transdermal glucose monitor therein.

2. The apparatus of claim 1, wherein the top wall has a circular shape.

3. The apparatus of claim 1, wherein the top wall has a uniform thickness.

4. The apparatus of claim 1, wherein the perimeter wall has a uniform thickness.

5. The apparatus of claim 1, wherein the perimeter wall is integrally formed with the top wall.

6. The apparatus of claim 1, wherein the perimeter wall extends perpendicularly from the top wall.

7. The apparatus of claim 1, wherein the perimeter wall comprises a resiliently stretchable material wherein the perimeter wall is configured to stretch to accommodate the transdermal glucose monitor.

8. The apparatus of claim 1, further comprising a plurality of protrusions coupled to and extending inwardly from the perimeter wall, the protrusions being positioned distally from the top wall, the protrusions being shaped and configured to seat on a shoulder of the transdermal glucose monitor at a bottom end thereof.

9. The apparatus of claim 8, wherein the protrusions are radially spaced from each other along the perimeter wall.

10. The apparatus of claim 8, wherein each protrusion tapers from the perimeter wall to a distal edge with respect to the perimeter wall.

11. The apparatus of claim 8, wherein each protrusion has a lower surface which is coplanar with a lower end of the perimeter wall.

12. A cover apparatus for a transdermal glucose monitor, the apparatus comprising:
a top wall having a circular shape, the top wall having a uniform thickness; and
a perimeter wall coupled to and extending away from the top wall, the perimeter wall and the top wall defining a cavity dimensioned and configured to receive the transdermal glucose monitor therein, the perimeter wall having a uniform thickness, the perimeter wall being integrally formed with the top wall, the perimeter wall extending perpendicularly from the top wall.

13. The apparatus of claim 12, wherein the perimeter wall comprises a resiliently stretchable material wherein the perimeter wall is configured to stretch to accommodate the transdermal glucose monitor.

14. The apparatus of claim 12, further comprising a plurality of protrusions coupled to and extending inwardly from the perimeter wall, the protrusions being positioned distally from the top wall, the protrusions being shaped and configured to seat on a shoulder of the transdermal glucose monitor at a bottom end thereof, the protrusions being radially spaced with respect to each other, each protrusion tapering from the perimeter wall to a distal edge with respect to the perimeter wall, each protrusion having a lower surface which is coplanar with a lower end of the perimeter wall.

15. A monitor covering assembly comprising:
a transdermal glucose monitor configured to attach to skin of a user; and
a cover removably mountable to the transdermal glucose monitor to surround exposed surfaces thereof, the cover comprising:
a top wall; and
a perimeter wall coupled to and extending away from the top wall, the perimeter wall and the top wall defining a cavity dimensioned to receive the transdermal glucose monitor therein.

16. The assembly of claim 15, wherein:
the transdermal glucose monitor has a disc shape; and
the top wall has a circular shape.

17. The assembly of claim 15, wherein:
the top wall having a uniform thickness; and
the perimeter wall having a uniform thickness.

18. The assembly of claim 15, wherein the perimeter wall is integrally formed with the top wall, the perimeter wall extending perpendicularly from the top wall.

19. The apparatus of claim 15, wherein the perimeter wall comprises a resiliently stretchable material wherein the perimeter wall is configured to stretch to accommodate the transdermal glucose monitor.

20. The apparatus of claim 15, further comprising a plurality of protrusions coupled to and extending inwardly from the perimeter wall, the protrusions being positioned distally from the top wall, the protrusions being shaped to be selectively seated on a shoulder of the transdermal glucose monitor at a bottom end thereof, the protrusions being radially spaced from each other along the perimeter wall, each protrusion tapering from the perimeter wall to a distal edge with respect to the perimeter wall, each protrusion having a lower surface which is coplanar with a lower end of the perimeter wall.
